(19) United States
(12) Patent Application Publication
(10) Pub. No.: US 2007/0141150 A1
(43) Pub. Date: Jun. 21, 2007

(54) Pharmaceutical composition

(76) Inventors: Raghupathi Kandarapu, Hyderabad (IN); Akhilesh Ashok Dixit, Nagpur (IN); Vijay Dinanathji Nasare, Nagpur Katol (IN); Mailatur Sivaraman Mohan, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 10/596,915

(22) PCT Filed: Dec. 23, 2004
(86) PCT No.: PCT/US04/43155

§ 371 Date: Jun. 29, 2006

(30) Foreign Application Priority Data
Dec. 30, 2003 (IN) 1065/CHE/2003

(51) Int. Cl. A61K 31/522; A61K 9/24; A61K 31/4172; A61K 31/401; A61K 31/198

(52) U.S. Cl. 424/472; 514/263.31; 514/423; 514/400; 514/561; 514/263.32

(57) ABSTRACT

A solid pharmaceutical dosage form comprises a core particle containing at least one pharmaceutical active ingredient and/or excipient that is sensitive to acid, a subcoating containing a chemically reactive component disposed over the core, and an enteric coating over the subcoating.

The present invention relates to pharmaceutical dosage forms comprising components that have a tendency to chemically interact with coatings having acidic functional groups, and more particularly to dosage forms containing components that interact with enteric coating materials.

Acidic polymers that have free carboxylic groups are widely used in pharmaceutical compositions, especially for enteric coating. Usually the purpose of an enteric coating is to prevent the degradation of the active ingredient under acidic conditions in the stomach. Another reason for using an enteric coat can be to delay and/or moderate the release of the active ingredient as the dosage form passes through the digestive tract.

The selection of an enteric coating becomes more critical when the active ingredient or an excipient in a composition has a tendency to chemically interact with enteric coating materials. Sometimes this results in formation of a slowly soluble or even insoluble coating that impairs the complete release of active ingredient from the dosage form. For example, some widely used antidepressant drugs such as fluoxetine, duloxetine, nortryptylline desipramine, sertralline, and paroxetine have been reported to chemically interact with enteric coating materials.

N. Sarisuta et al., “Physico-chemical Characterization of Interactions Between Erythromycin and Various Film Polymers,” International Joumal of Pharmaceutics, Vol. 186, pages 109-118, 1999, prepared films of various acidic polymers containing erythromycin and determined an amine salt interaction between the carboxyl group of the acid polymers and the nitrogen atom of erythromycin, using a nuclear magnetic resonance technique. The solid solution of erythromycin in all of the polymer films was found to be physically stable.

S. Takka et al., “Effect of Anionic Polymers on the Release of Propranolol Hydrochloride from Matrix Tablets,” European Journal of Pharmaceutics and Biopharmaceutics, Vol. 52, pages 75-82, 2001, utilized an interaction between propranolol hydrochloride and anionic polymers to control the release of propranolol hydrochloride from hydroxypropyl methylcellulose matrices. The interactions between propranolol hydrochloride and anionic polymers were confirmed by an ultraviolet difference spectra method.

H. K. Lee et al., “Propranolol:Methacrylic Acid Copolymer Binding Interaction,” Journal of Pharmaceutical Science, Vol. 80, pages 178-180, 1991, observed that the polyanionic form of methacrylic acid/methacrylic acid methyl ester copolymer reacts readily with propranolol hydrochloride to give a sparingly soluble complex at saturation equilibrium. The propranololi/methacrylic acid copolymer complex was characterized by differential thermal analysis, and infrared and ultraviolet spectroscopic methods. The propranolol content was found to be 68% in the complex. The value of the Hill coefficient (1.5) indicated that there is a high degree of positive cooperative interaction between propranolol and the polymer.

In the past, several approaches were developed to address the problem, such as building a strong cementing layer or subcoat between the enteric coating material and the acid sensitive core, whereby interacting components are physically separated, or by mixing an alkaline or buffering substance with the components of the drug-containing core. In the process of preventing an interaction between the enteric coat and the reactive components in the core, water soluble subcoating materials were utilized to prevent delays in release of drug under intestinal pH conditions. Even though workers have managed to develop enteric coated pharmaceutical compositions of such active ingredients or excipients, the problem of chemical interaction and the chemistry involved in such interactions is not well understood.

U.S. Pat. No. 5,910,319 discloses a means for preventing chemical interactions, wherein an enteric coating material (hydroxypropyl methylcellulose acetate succinate, or “HPMCAS”), which contains not less than 4% and not more than 28% of succinyl groups, is neutralized with ammonia prior to use. The patent teaches neutralization of carboxylic groups in the enteric polymer only to an optimum level, whereby an interaction of active ingredient with enteric coating is prevented and the acid resistance of the enteric coating was not affected. However, any chemical modification to the polymer precursor is likely to affect the physical properties of the final polymer and possibly degrade the structural integrity of the dosage form.

U.S. Pat. No. 4,786,505 describes a method of preparing a stable formulation of the acid-sensitive drug omeprazole, wherein a water-soluble intermediate coat is layered onto the drug-containing core surface. An enteric coating is then layered over the water-soluble intermediate layer.

U.S. Pat. No. 5,035,899 discloses a formulation of acid labile benzimidazole derivatives, having a subcoating of slightly water-soluble film-forming material and fine particles of a slightly water-soluble substance, suspended in the coating material.

In U.S. Pat. No. 5,035,899 Saeki teaches the development of a pharmaceutical composition by coating a core comprising a benzimidazole derivative using a slightly water-soluble, film-forming material comprising ethyl cellulose and polyvinyl acetate, then applying an enteric coat.

These approaches have not been found entirely suitable for preventing chemical interactions between acid-sensitive components and the enteric coating, while preserving the acid-resistance of the coating and not changing drug release characteristics at higher pH conditions.

### SUMMARY OF THE INVENTION

The present invention provides, in one aspect, enteric coated pharmaceutical compositions comprising a drug and/or an excipient having a tendency to chemically interact with enteric coating material. The pharmaceutical composition comprises:

(1) a core comprising one or more drugs and one or more pharmaceutically acceptable excipients;

(2) a subcoat or intermediate layer, comprising at least one chemically reactive substance;

(3) an enteric layer comprising an enteric coating material; and

(4) optionally, a finishing coat.

In another aspect, the invention provides a solid dosage form comprising:

(1) a core comprising an acid-sensitive pharmaceutically active ingredient, an acid-sensitive excipient, or both;

(2) a subcoating upon the core comprising a substance that reacts with acidic functional groups; and

(3) an acidic functional group-containing enteric coating over the subcoating.

In yet another aspect, the invention provides a solid dosage form comprising:

(1) a core comprising an acid-sensitive pharmaceutically active ingredient having an amino group;

(2) a subcoating upon the core comprising an α-amino acid; and

(3) an acidic functional group-containing component in an enteric coating over the subcoating.

In a further aspect, the invention provides a solid dosage form comprising:

(1) a core comprising an acid-sensitive pharmaceutically active ingredient having an amino group;

(2) a subcoating upon the core comprising glycine; and

(3) an acidic functional group-containing component in an enteric coating over the subcoating.

### DETAILED DESCRIPTION

For purposes of this invention, an “enteric coating” is a hydrophobic layer that preferably completely surrounds inner components of a pharmaceutical dosage form, which inner components include at least a portion of the total active drug ingredient or ingredients. The enteric coating resists decomposition by gastric juices and thereby protects inner components from interaction with acidic substances that are present in the stomach. Frequently, the enteric coating is itself an acidic substance and will be decomposed or removed upon exposure to a less acidic environment. Various commercially available enteric coating materials are designed to undergo decomposition or removal when exposed to certain pH regimes that are associated with specific regions in the digestive tract of a human: pH between about 3.5 and 6 in the duodenum; pH about 6-7 in the jejunum; pH about 7-7.5 in the ileum; and pH about 7-8 in the colon. In most instances complete removal of the enteric coating is not necessary for obtaining a desired drug release, as any substantial discontinuity in the coating will permit fluid ingress for interaction with the inner components.

As discussed above, the commonly used enteric coating materials are polymers having acidic functional groups and therefore are capable of reacting with substances that have a more basic character. These reactions sometimes occur in the solid state and thereby directly affect the stability of a pharmaceutical dosage form that contains a basic drug substance or excipient as an inner (core) component, in contact with an enteric coating. For example, dosage form stability can be decreased during storage by a reaction that renders a portion of the drug substance inert, or by affecting the integrity of the enteric coating so that it does not adequately resist stomach acid.

For purposes of this invention, the term “chemically reactive substance,” “reactive substance,” or “chemically reactive component” represents a component of a pharmaceutical dosage form that undergoes a competitive chemical reaction with carboxylic groups in the enteric coating to prevent an interaction between inner components of the dosage form and the enteric coating polymer. Chemically reactive substances are present in a subcoating upon a core, or in both the core and a subcoating, the subcoating being in contact with an enteric coating.

A pharmaceutical dosage form of this invention comprises at least: (1) a core containing one or more drug substances; (2) a chemically reactive substance contained in a subcoating over the core; and (3) an enteric coating, layered over the subcoating. Sometimes the dosage form has additional layers, such as those dosage forms where a drug substance that is not acid-sensitive will be present in a film or other coating that is applied over the enteric coating, or dosage forms where a drug substance is applied as a layer onto a physiologically inert nonpariel or other particle, and this is considered to constitute the core. Components of the core are sometimes referred to herein as “inner components.”

Core

The core is prepared by applying a drug-containing layer to an inert particle, or it can comprise a particle comprising a drug and at least one pharmaceutically acceptable excipient. The core comprises a drug substance that is chemically reactive with an enteric coating and/or a chemically reactive excipient substance.

Useful drug substances for the purposes of this invention include those having one or more functional groups that tend to react with functional groups present in an enteric coating. Typically, but not exclusively, the drug substance functional groups that are reactive will be primary, secondary, or tertiary amino groups; representative examples of such drug substances are, without limiting the invention thereto: benzimidazole proton pump inhibitors such as esomeprazole, lansoprazole, pantoprazole, omeprazole and rabeprazole; antihistamines such as chlorpheniramine; anti-infectives such as erythromycin; adrenergic antagonists such as propranolol and atenolol; and xanthines such as theophylline; including their salts, esters, hydrates, and other pharmaceutically useful forms as may be applicable to a particular drug substance.

The invention is particularly useful for preparing dosage forms that contain an antidepressant drug, including amitryptyline, amoxapine, bupropion, desipramine, doxepin, duloxetine, fluoxetine, fluvoxamine, imipramine, maprotiline, mirtazapine, nefazodone, nortriptyline, paroxetine, pheneizine, protriptyline, sertraline, tranylcypromine, trazodone, trimipramine, and venlafaxine, or any pharmaceutically acceptable salt thereof.

In some instances, the drug substance will not have a reactive functional group, but an excipient that is desired for incorporation into the core will contain a reactive functional group. In this situation, any drug substance that can be formulated into a solid dosage form is useful for the present invention. Examples of such excipients include: albumin, aspartame, benzalkonium chloride, benzethonium chloride, cetrimide, chitosan, chlorhexidine, denatonium benzoate, diethanolamine, edetic acid and its salts, ethanolamine, gelatin, hexetidine, imidurea, lecithin, meglumine, monosodium glutamate, saccharin, and others.

The invention is also useful when both the drug substance and one or more excipients in the core are capable of undergoing an undesired reaction with enteric coating materials.

The core may be in any of a number of physical forms, such as pellets, granules, beads, minitablets and tablets, such as are conventionally used for the oral administration of pharmaceutical substances. Pellets can be prepared using techniques such as extrusion and spheronisation, by coating nonpareil seeds, by melt pelletization, or by another conventional pelletization processes. Useful nonpareil seeds can be prepared from starch and sucrose, using techniques that are well known in the art. Tablets and minitablets can be prepared by customary compression techniques with or without involving a prior granulation step, such as wet granulation, dry granulation, or melt granulation. Minitablets, granules, pellets, beads, and the like that are prepared according to this invention can be filled into capsules to produce a final dosage form, for ease of administration.

The core of a formulation is normally prepared by mixing active ingredient or ingredients with a desired combination of excipient ingredients such as fillers, surfactants, disintegrants, binders, lubricants, and optionally a chemically reactive agent. When a chemically reactive agent is present in the core, its concentration typically will be about 0.5 to about 20 percent, or about 1 to about 15 percent, or about 2 to about 8 percent, of the total weight of the core.

In general, the core comprises about 10 to about 80 percent of the total dosage form weight, or about 30 to about 70 percent, or about 45 to about 65 percent.

Subcoating

The purpose of the subcoating is to inhibit chemical interactions between reactive inner, or core, components and enteric polymers, and to provide a smooth base for the deposition of an enteric coating. A subcoating of this invention comprises a chemically reactive component that can rapidly react with surface functional groups of the enteric coating, preferably forming water-soluble reaction products.

Suitable chemically reactive components include amino acids, particularly but not limited to the naturally occurring a-amino acids that are recognized as being safe for ingestion. Examples of those amino acids are glycine, alanine, valine, leucine, isoleucine, serine, threonine, methionine, cysteine, aspartic acid, asparagine, glutamic acid, glutamine, arginine, lysine, histadine, phenylalanine, tyrosine, tryptophan, and proline. In general, amino acids having lower molecular weights are preferred. Glycine, for example, has been found particularly useful in the invention.

The chemically reactive component can conveniently be included in an aqueous-based subcoating comprising a cellulosic polymer or derivative thereof, which is conventionally used in the pharmaceutical industry for forming water-soluble or water-dispersible films. Some preferred polymers are, without limitation thereto, methylcellulose, ethylcellulose, hydroxypropyl methylcellulose, hydoxypropyl cellulose, and combinations thereof. Acrylics, such as methacrylate and methyl methacrylate copolymers, and vinyls, such as polyvinyl alcohol, can be used for the subcoating. Other polymers, such as poly-N-vinyl-2-pyrrolidone (“povidone”) and its derivatives such as copolymers of N-vinyl-2-pyrrolidone and vinyl acetate (“copolyvidonum”), are also useful.

Frequently, other materials such as plasticizers will be included to improve the properties of the coating; representative plasticizers are glycerin, propylene glycol, a polyethylene glycol, acetylated monoglyceride, a citrate ester, a phthalate ester, and dibutyl subacetate.

Typically, the subcoating will comprise about 1 to about 20 percent, or about 5 to about 15 percent, or about 7 to about 12 percent of the total weight of the final coated dosage form. The amount of chemically reactive component incorporated into the subcoating is typically about 10 to about 60 percent, or about 15 to about 30 percent, by weight of the total subcoating; expressed as a percentage of the final dosage form, the chemically reactive component therefore typically comprises about 0.1 to about 12 weight percent, or about 0.5 to about 9 percent, or about 0.7 to about 7 percent by weight of the dosage form. The amount of subcoating will depend somewhat on the nature of the core particles, as smaller forms such as pellets and granules usually will have a larger total surface area and require a larger quantity of coating material to obtain coverage, than will tablets. For this reason, the percentages above are only general guidelines.

The subcoating may further include other commonly used functional ingredients such as fillers, glidants or antiadherents such as talc or fumed silica, and the like.

Application of the subcoating to the particles proceeds according to accepted practice, such as by spraying onto particles present in a rotating pan, using a fluidized bed coating apparatus, and the like. The subcoating should be at least substantially continuous over the core, preferably having no discontinuities that could permit direct physical contact between the core and an enteric coating.

Another useful subcoating material is zein, a prolamine that is insoluble in both water and alcohol, but soluble in mixtures thereof. The chemically reactive component is dissolved in a zein solution, and applied to core particles in the usual manner. Zein, derived from corn and in grades acceptable for pharmaceutical uses, is commercially available from Freeman Industries LLC of Tuckahoe, N.Y. U.S.A. as Zein F4000 and Zein F6000. The zein can be used alone, or in combination with a polymer or mixture of polymers.

Enteric Coating

An enteric coating is an element of the pharmaceutical dosage form of the present invention. One advantage of the present invention is the ability to use virtually any polymer, or this coating, as compared with the teaching of U.S. Pat. No. 5,910,319 that the concentration of acidic groups in the coating must be limited. In the present invention, coatings that contain much higher amounts of acidic groups can be used.

Examples of useful enteric coating materials are the polymethacrylates sold by Röhm GmbH & Co. KG, Darmstadt, Germany using the EUDRAGIT trademark. The EUDRAGIT L 100-55 and L 30 D-55 products are 1:1 copolymers of methacrylic acid and ethyl acrylate that dissolve at pH values above about 5.5. EUDRAGIT L 100 is a 1:1 copolymer of methacrylic acid and methyl methacrylate that dissolves at pH above about 6. Mixtures of EUDRAGIT L 100 and EUDRAGIT S 100, a 1:2 copolymer of methacrylic acid and methyl methacrylate, dissolve at pH above about 6 to 6.5. EUDRAGIT S 100 alone dissolves at pH above about 6.5-7.5. Chemically related enteric coating polymers are also sold by Eastman Chemical Company of Kingsport, Tennessee USA under the trademark EASTACRYL, and by BASF of Ludwigshafen, Germany under the trademark KOLLICOAT.

“HPMCP” (hydroxypropyl methylcellulose phthalate, or hypromellose phthalate), is another useful enteric coating polymer. This material is almost universally accepted by regulatory authorities as an enteric coating substance. By varying the phthalate content, the pH for dissolution of a coating can be controlled, generally to values between about 5 and about 5.5. An example of a useful HPMCP is sold by Shin-Etsu Chemical Co., Ltd. of Tokyo, Japan as HP-55; this product has about 31 percent phthalyl content and is soluble in Mcllvaine's buffer solution of pH 5.5 or greater. Also available from this source is the HP-50 product that has a phthalyl content about 24 percent, and solubility at pH 5.0 and above.

Other types of enteric coating polymers are known, and can be used for purposes of the invention. These include polyvinyl acetate phthalate, cellulose acetate trimellitate, carboxymethyl ethylcellulose, hydroxymethylcellulose acetate succinate, and cellulose acetate phthalate. The polymers are commercially available, from several sources.

Many of the commercial products are sold as ready-to-use mixtures of the polymer, a plasticizer, and other desired functional components such as glidants and/or pigments. These products can be simply dissolved or dispersed in a suitable solvent (if not supplied in a fluid form) and applied to particles containing the drug substance.

The enteric coating may be applied as a powder or from an aqueous or organic solution or dispersion. It generally is preferred to coat the subcoated core pellets, granules, tablets, etc. with a solution that contains the enteric polymer, since this facilitates a uniform coating. The enteric coating layer is applied to the subcoated cores using conventional coating techniques such as pan coating or fluidized bed coating using solutions of any of the previously mentioned polymers, and others. In the finished dosage form, the quantity of enteric coat is typically about 5 to about 30 percent of the total weight, or about 10 to about 25 percent. Commonly used plasticizers, such as triethyl citrate, and solid property-modifying components such as talc can also be incorporated into an enteric coating solution, as is well-known in the art.

The enteric coating solution is typically applied as a solution of enteric polymer in organic solvents like acetone, dichloromethane and isopropyl alcohol, and combinations thereof. In another embodiment of the invention, a suspension of enteric coated polymer can be applied over the subcoat, provided the suspension remains homogenous. Application of the enteric layer to the subcoated product can be conducted using fluid bed type equipment or in a conventional rotating coating pan with simultaneous spraying of enteric polymer solution or suspension and warm air drying. Temperature of the drying air and the temperature of the circulating mass of pellets should be kept in the ranges advised by the manufacturer of the particular enteric polymer being used.

A finishing layer over the enteric layer is not necessary in every case, but generally improves the elegance of the product and its handling, storage and flow characteristics and may provide further benefits as well. The simplest finishing layer is simply a small amount, such as about 1% by weight, of an anti-static ingredient such as talc or silicon dioxide, simply dusted on the surface of the pellets.

As is known in the art, the finishing coating for dosage forms such as tablets can be a film coating that improves the surface properties and facilitates imprinting of identifying information. Although less preferable from the manufacturing convenience perspective, a sugar coating that is applied in a usual manner can be used for the same purposes.

Following are examples of pharmaceutical compositions incorporating active ingredients that are sensitive to enteric polymers. The examples are intended only to illustrate the invention, and are not to be construed to limit the scope of the invention as defined by the appended claims. In the examples, solvents that are used in the procedures but are not present in the final dosage form are not included in ingredients listings.

### EXAMPLE 1

The following ingredients and procedure were used to prepare delayed release fluoxetine capsules containing coated pellets, according to the present invention.

| | | |
|---|---|---|
| INGREDIENTS | FUNCTIONAL CATEGORY | MG/CAPSULE |

CORE PELLETS

| | | |
|---|---|---|
| Fluoxetine hydrochloride | Active agent | 101 |
| Microcrystalline | Filler | 7.5 |
| cellulose* | | |
| Mannitol | Filler | 212.7 |
| Glycine | Stabilizer | 20 |
| Sodium lauryl sulfate | Solubilizer | 11.3 |
| Fumed silica | Dispersing aid | 7.5 |
| Copovidone** | Binder | 15 |
| Core weight | | 375 |

SUBCOATING

| | | |
|---|---|---|
| Copovidone** | Polymer | 20 |
| Glycine | Reactive substance | 10 |
| Talc | Anti-adherent | 5 |
| Subcoat Weight | | 35 |

ENTERIC COATING

| | | |
|---|---|---|
| HPMCP (HP-55) | Enteric polymer | 94.2 |
| Triethyl citrate | Plasticizer | 9.4 |
| Talc | Anti-adherent | 14.1 |
| Enteric Coat Weight | | 117.7 |

*AVICEL ™ PH 105, sold by FMC Corporation, Philadelphia, Pennsylvania U.S.A.
**PLASDONE ™ S-630, sold by International Specialty Products, Wayne, New Jersey U.S.A.

A. Preparation of core pellets
   i) The core ingredients, except for copovidone, were weighed into a polyethylene bag and mixed for 5 minutes.
   ii) The blend was passed through a sieve having #40 mesh openings.
   iii) The sieved blend was placed into a double cone blender and mixed for 15 minutes.
   iv) Copovidone was dissolved in water and used as a granulating fluid to granulate the blend.
   v) The wet granulated mass was subjected to an extrusion-spheronization process to produce pellets.
   vi) Pellets were dried at 65° C. for 3 hours in a tray dryer.
   vii) Pellets passing through a sieve having #14 openings, but retained on a sieve having #20 openings, were selected for use in the subcoating procedure.
B. Subcoating
   i) The subcoating formula ingredients were mixed with water to dissolve soluble components.
   ii) Pellets from the procedure above were loaded into a Neocota coating apparatus and coated with the subcoating composition to achieve a 9-10 percent weight gain.
   iii) Residual water was removed by drying at an elevated temperature.
C. Enteric coating and filling into capsules
   i) The enteric coating ingredients were dissolved using isopropyl alcohol and dichloromethane (1:1 by weight) as a solvent.
   ii) Subcoated pellets from the procedure above were loaded in the Neocota coating apparatus and coated with the enteric coating solution to achieve a 23-25 percent weight gain.
   iii) After removing solvent by drying at an elevated temperature, the enteric coated pellets were mixed with 1 percent by weight talc and filled into size 0 elongated hard gelatin capsules.

The enteric coated pellets exhibit acid resistance to gastric conditions at 37° C. Using dissolution test apparatus and conditions described in United States Pharmacopeia 24, after two hours the quantity of active ingredient released in acidic gastric media was found to be below 10%, while the same composition in a buffer solution of pH 6.8, maintained at a temperature of 37° C., released not less than 80% of active ingredient after 10-30 minutes.

### EXAMPLE 2

Esomeprazole-containing tablets are prepared, using the following ingredients and procedure:

| | |
|---|---|
| INGREDIENTS | MG/TABLET |

CORE

| | |
|---|---|
| Esomeprazole magnesium trihydrate | 44.5 |
| Magnesium oxide | 20 |
| Copovidone | 17.5 |
| Crospovidone | 10 |
| Mannitol | 227 |
| Colloidal silicon dioxide | 3.5 |
| Sodium stearyl fumarate | 17.5 |
| Core Weight | 340 |

SUBCOATING

| | |
|---|---|
| DL-Alanine | 5 |
| Zein 6000 | 10 |
| Subcoat Weight | 15 |

ENTERIC COATING

| | |
|---|---|
| EUDRAGIT L100-55 | 19.1 |
| Triethyl citrate | 1.9 |
| Titanium dioxide | 3.8 |
| Talc | 2.9 |
| Enteric Coat Weight | 27.7 |

Esomeprazole magnesium trihydrate, magnesium oxide, copovidone, crospovidone, mannitol, and silicon dioxide are blended, then sodium stearyl fumarate is added with further blending. This mixture is directly compressed into core tablets. Alanine is dissolved in water, an equal volume of ethanol is added, then zein is added and dissolved with stirring; the tablets are subcoated in a rotating pan with the aqueous alcoholic solution of alanine and zein, then dried. Finally, the enteric coating ingredients are dispersed in water and coated onto the subcoated tablets, followed by a final drying.

### EXAMPLE 3

Rabeprazole sodium tablets are prepared using the following components and procedure:

| | |
|---|---|
| INGREDIENTS | MG/TABLET |

CORE

| | |
|---|---|
| Rabeprazole sodium | 20 |
| Mannitol | 97.01 |
| Low-substituted hydroxypropyl | 14.4 |
| cellulose (“L-HPC”) | |
| Magnesium oxide | 40 |
| Sodium lauryl sulfate (“SLS”) | 1.8 |
| Hydroxypropyl methylcellulose, | 3 |
| 5 mPa · s (“HPMC”) | |
| Talc | 1.54 |
| Magnesium stearate | 2.25 |
| Core Weight | 180 |

SUBCOATING

| | |
|---|---|
| Glycine | 5 |
| Zein 6000 | 4.9 |
| Triethyl citrate | 0.49 |
| Subcoat Weight | 10.39 |

ENTERIC COATING

| | |
|---|---|
| EUDRAGIT L100-55 | 14.46 |
| Triethyl citrate | 1.44 |
| Talc | 0.79 |
| Enteric Coat Weight | 16.69 |

FILM COATING

| | |
|---|---|
| OPADRY ™ Yellow OY-52945 | 5.05 |

PRINTING

| | |
|---|---|
| OPACODE ™ Black | q.s. |

Magnesium oxide is sifted through a 60 mesh sieve. Rabeprazole sodium, L-HPC, mannitol and the sifted magnesium oxide are sifted through a 40 mesh sieve. The materials are then mixed for 30 minutes in a mixer-granulator. SLS is dissolved in water and HPMC is dissolved in warm water. The rabeprazole sodium mixture is combined with the SLS and HPMC granulating solutions. The wet mass is dried in a fluid bed drier and the dried granules are sifted through a 20 mesh sieve. The sifted granules are blended with L-HPC in a double cone blender for 5 minutes. Magnesium stearate (sifted through a 60 mesh sieve) is added to the blend and mixed for 5 minutes. The lubricated blend is then compressed into core tablets.

Glycine is dissolved in water, an equal volume of ethanol is added, and the zein is dissolved in the solution. The core tablets are subcoated with the water-alcohol coating solution, and dried. The subcoated tablets are further coated with the aqueous enteric coating dispersion, and dried. The enteric coated tablets are additionally film-coated with OPADRY solution, and dried. Finally, the coated tablets are imprinted using OPACODE black ink.

The OPADRY product is a dry powder sold by Colorcon, West Point, Pa. U.S.A., containing a polymers plasticizer, and pigment, that is mixed with water and sprayed onto tablets or other solid dosage forms. This film coating procedure and many alternative film coating products are well known in the art. The OPACODE ink is sold by this same supplier.

1. A solid dosage form comprising:
(a) a core comprising an acid-sensitive pharmaceutically active ingredient, an acid-sensitive excipient, or both;
(b) a subcoating upon the core comprising a substance that reacts with acidic functional groups; and
(c) an acidic functional group-containing enteric coating over the subcoating.

2. The solid dosage form of claim 1, wherein the pharmaceutically active ingredient has an amino group.

3. The solid dosage form of claim 1, wherein the substance that reacts with acidic functional groups comprises an amino acid.

4. The solid dosage form of claim 1, wherein the substance that reacts with acidic functional groups comprises an α-amino acid.

5. The solid dosage form of claim 1, wherein the substance that reacts with acidic functional groups is selected from glycine, alanine, valine, leucine, isoleucine, serine, threonine, methionine, cysteine, aspartic acid, asparagine, glutamic acid, glutamine, arginine, lysine, histadine, phenylalanine, tyrosine, tryptophan, and proline.

6. The solid dosage form of claim 1, wherein the substance that reacts with acidic functional groups comprises about 0.1 to about 12 percent of the dosage form weight.

7. The solid dosage form of claim 1, wherein the substance that reacts with acidic functional groups comprises about 0.5 to about 9 percent of the dosage form weight.

8. The solid dosage form of claim 1, wherein the substance that reacts with acidic functional groups comprises about 0.7 to about 7 percent of the dosage form weight.

9. The solid dosage form of claim 2, wherein the acid sensitive pharmaceutically active ingredient comprises an antihistamine, an anti-infective, an adrenergic antagonist, or a xanthine.

10. The solid dosage form of claim 2, wherein the acid-sensitive pharmaceutically active ingredient comprises an antidepressant.

11. The solid dosage form of claim 2, wherein the acid-sensitive pharmaceutically active ingredient comprises a benzimidazole proton pump inhibitor.

12. A solid dosage form comprising:
(a) a core comprising an acid-sensitive pharmaceutically active ingredient having an amino group;
(b) a subcoating upon the core comprising an α-amino acid; and
(c) an acidic functional group-containing component in an enteric coating over the subcoating.

13. The solid dosage form of claim 12, wherein the substance that reacts with acidic functional groups is selected from glycine, alanine, valine, leucine, isoleucine, serine, threonine, methionine, cysteine, aspartic acid, asparagine, glutamic acid, glutamine, arginine, lysine, histadine, phenylalanine, tyrosine, tryptophan, and proline.

14. The solid dosage form of claim 12, wherein a reaction product of the α-amino acid with the acidic functional group-containing component is water-soluble.

15. The solid dosage form of claim 12, wherein the amino acid comprises about 0.1 to about 12 percent of the dosage form weight.

16. The solid dosage form of claim 12, wherein the amino acid comprises about 0.5 to about 9 percent of the dosage form weight.

17. The solid dosage form of claim 12, wherein the amino acid comprises about 0.7 to about 7 percent of the dosage form weight.

18. A solid dosage form comprising:
(a) a core comprising an acid-sensitive pharmaceutically active ingredient having an amino group;
(b) a subcoating upon the core comprising glycine; and
(c) an enteric coating, comprising an acidic functional group-containing component, over the subcoating.

19. The solid dosage form of claim 18, wherein the acid-sensitive pharmaceutically active ingredient comprises an antidepressant.

20. The solid dosage form of claim 18, wherein the acid-sensitive pharmaceutically active ingredient comprises a benzimidazole proton pump inhibitor.
